(19) United States
(12) Patent Application Publication
(10) Pub. No.: US 2014/0094485 A1
(43) Pub. Date: Apr. 3, 2014

(54) SOLID STATE FORMS OF HIV INHIBITOR

(71) Applicant: Gilead Sciences, Inc., Foster City, CA (US)

(72) Inventors: Brandon H. Brown, Burlingame, CA (US); Ernest Anthony Carra, Foster City, CA (US); Xiang Wang, Foster City, CA (US)

(21) Appl. No.: 14/044,193

(22) Filed: Oct. 2, 2013

Related U.S. Application Data
(60) Provisional application No. 61/744,868, filed on Oct. 3, 2012.

(51) Int. Cl. A61K 31/4741; A61K 45/06; C07D 491/06

(52) U.S. Cl. CPC A61K 31/4741; C07D 491/06; A61K 45/06
USPC 514/291; 546/89

(57) ABSTRACT

The invention relates to a novel hemi-succinate salt form of (2S)-2-tert-butoxy-2-(4-(2,3-dihydropyrano[4,3,2-de]quinolin-7-yl)-2-methylquinolin-3-yl)acetic acid and a novel crystalline form thereof, methods for the preparation thereof, pharmaceutical compositions thereof and their use in the treatment of Human Immunodeficiency Virus (HIV) infection.

### CROSS-REFERENCE TO RELATED APPLICATION

This application claims the benefit under 35 U.S.C. §119(e) of U.S. Provisional Patent Application No. 61/744,868, filed Oct. 3, 2012, which application is incorporated herein by reference in its entirety.

### BACKGROUND

1. Field

This invention relates to a novel hemi-succinate salt form of Compound (I) and a novel crystalline form of the hemi-succinate salt of Compound (I) as described herein, methods for the preparation thereof, pharmaceutical compositions thereof, and their use in the treatment of Human Immunodeficiency Virus (HIV) infection.

2. Description of the Related Art

Compound (I), (2S)-2-tert-butoxy-2-(4-(2,3-dihydropyrano[4,3,2-de]quinolin-7-yl)-2-methylquinolin-3-yl)acetic acid, is an HIV non-catalytic site integrase inhibitor.

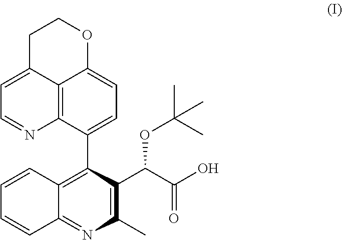

Compound (I) falls within the scope of the HIV inhibitors disclosed in WO 2007/131350. Compound (I) is disclosed specifically as compound no. 1144 in WO 2009/062285. Compound (I) can be prepared according to the general procedures found in WO 2007/131350 and WO 2009/062285, which are hereby incorporated by reference.

In drug development, it is necessary to produce a compound that can enable formulation to meet targeted pharmaceutical requirements and specifications. This is typically achieved through the use of a stable crystalline form of the drug. It is desirable to select a drug form that is easily and consistently manufactured and may be produced on a large-scale in a cost-efficient manner. The present invention fulfills these needs and provides further related advantages.

### BRIEF SUMMARY

The present invention provides a novel hemi-succinate salt form of Compound (I) and a novel crystalline form of the hemi-succinate salt of Compound (I) which are useful in the treatment of an HIV infection.

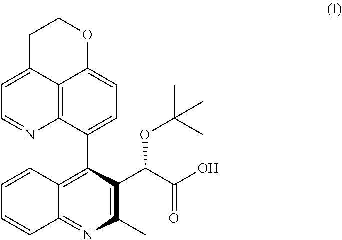

Further objects of this invention arise for the one skilled in the art from the following description and the examples.

In one embodiment, the invention is directed to a hemi-succinate salt of Compound (I):

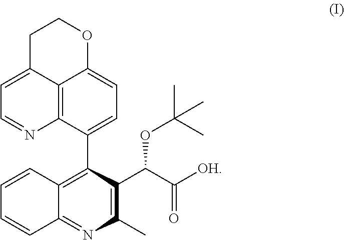

The above hemi-succinate salt form of Compound (I) may be in a non-crystalline or crystalline state, each of which may exist as a solvate or non-solvate.

In a further embodiment of the invention, the hemi-succinate salt of Compound (I) is in crystalline Form A.

A further embodiment of the invention is a crystalline hemi-succinate salt of Compound (I) in crystalline Form A having an X-ray powder diffraction pattern comprising peaks at 7.1, 10.3 and 12.5 degrees 2θ (±0.2 degrees 2θ) when measured using CuKα radiation.

A further embodiment of the invention is a crystalline hemi-succinate salt of Compound (I) in crystalline Form A having an X-ray powder diffraction pattern comprising peaks at 7.1, 10.3 and 12.5 degrees 2θ (±0.2 degrees 2θ) and further comprising peaks at 18.9, 20.1 and 25.1 degrees 2θ (±0.2 degrees 2θ) when measured using CuKα radiation.

A further embodiment of the invention is a crystalline hemi-succinate salt of Compound (I) in crystalline Form A having an X-ray powder diffraction pattern comprising peaks at 7.1, 10.3, 12.5, 18.9, 20.1 and 25.1 degrees 2θ (±0.2 degrees 2θ) and further comprising peaks at 9.0, 22.8, 26.1 and 29.9 degrees 2θ (±0.2 degrees 2θ) when measured using CuKα radiation.

A further embodiment of the invention is a crystalline hemi-succinate salt of Compound (I) in crystalline Form A having an X-ray powder diffraction pattern substantially the same as that shown in FIG. 1.

A further embodiment of the invention is a crystalline hemi-succinate salt of Compound (I) in crystalline Form A having a DSC thermogram substantially the same as that shown in FIG. 2.

A further embodiment of the invention is a crystalline hemi-succinate salt of Compound (I) in crystalline Form A having a TGA curve substantially the same as that shown in FIG. 4.

A further embodiment of the invention is a crystalline hemi-succinate salt of Compound (I) in crystalline Form A having an X-ray powder diffraction pattern comprising peaks at 7.1, 10.3 and 12.5 degrees 2θ (±0.2 degrees 2θ) when measured using CuKα radiation and having a DSC thermogram substantially the same as that shown in FIG. 2.

A further embodiment is a crystalline hemi-succinate salt of Compound (I) in crystalline Form A having an XRPD pattern comprising peaks at 7.1, 10.3 and 12.5 degrees 2θ (±0.2 degrees 2θ) as described above and also exhibiting a TGA curve substantially the same as that shown in FIG. 4.

Another embodiment of the invention is a pharmaceutical composition comprising a hemi-succinate salt of Compound (I) as described above and at least one pharmaceutically acceptable carrier or diluent.

Another embodiment of the invention is a pharmaceutical composition as described above further comprising at least one other antiviral agent.

Another embodiment of the invention is the use of Compound (I) as described above or a pharmaceutical composition as described above for the treatment of an HIV infection in a human having or at risk of having the infection.

Another embodiment of the invention involves a method of treating or preventing an HIV infection in a human having or at risk of having the infection by administering to the human a therapeutically effective amount of a hemi-succinate salt of Compound (I) as described above, or a pharmaceutical composition as described above comprising the hemi-succinate salt of Compound (I), alone or in combination with at least one other antiviral agent, administered together or separately.

Also within the scope of this invention is the use of a hemi-succinate salt of Compound (I), as described herein, for the manufacture of a medicament for the treatment or prevention of an HIV infection in a human.

Another embodiment of this invention is a process to prepare crystalline Form A of the hemi-succinate salt of Compound (I) comprising the following steps:
(i) dissolving Compound (I) in a suitable solvent to obtain a mixture;
(ii) slowly heating the mixture of step (i) with stirring to a temperature to obtain a solution or slurry;
(iii) slowly adding succinic acid to the solution or slurry of step (ii) to obtain a solution;
(iv) optionally slowly adding a solution of sodium hydroxide to the solution of step (iii);
(v) optionally slowly cooling the solution of the preceding step;
(vi) optionally adding a seed comprising a hemi-succinate salt of Compound (I) to the solution of the preceding step to obtain a slurry;
(vii) optionally slowly adding a solution of sodium hydroxide to the solution or slurry of the preceding step;
(viii) cooling the mixture of the preceding step; and
(ix) collecting the solid material obtained in step (viii) to obtain the hemi-succinate salt of Compound (I).

Further embodiments of this invention arise for the one skilled in the art from the following description and the examples.

### BRIEF DESCRIPTION OF THE DRAWINGS

FIG. 1 is the X-ray powder diffraction (XRPD) pattern of the hemi-succinate salt of Compound (I), Form A.

FIG. 2 is the differential scanning calorimetry (DSC) thermogram of the hemi-succinate salt of Compound (I), Form A (onset is 159.3° C.).

FIG. 3 is the single crystal structure of the hemi-succinate salt of Compound (I), Form A.

FIG. 4 is the thermal gravimetric analysis (TGA) curve of the hemi-succinate salt of Compound (I), Form A.

FIG. 5 shows the tabletability of an immediate release formulation of the hemi-succinate salt of Compound (I), Form A.

### DETAILED DESCRIPTION

### Definitions

Terms not specifically defined herein should be given the meanings that would be given to them by one of skill in the art in light of the disclosure and the context. As used throughout the present application, however, unless specified to the contrary, the following terms have the meaning indicated:

Compound (I), (2S)-2-tert-butoxy-2-(4-(2,3-dihydropyrano[4,3,2-de]quinolin-7-yl)-2-methylquinolin-3-yl)acetic acid:

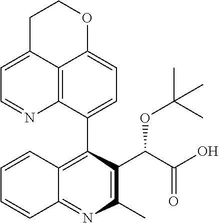

may alternatively be depicted as:

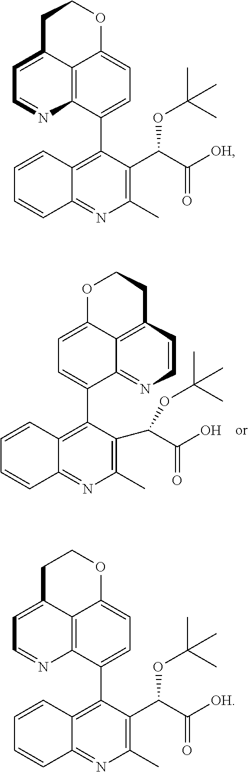

In addition, as one of skill in the art would appreciate, Compound (I) may alternatively be depicted in a zwitterionic form.

The term “solvate” refers to a crystalline solid containing amounts of a solvent incorporated within the crystal structure. As used herein, the term “solvate” includes hydrates.

The term “non-solvate” refers to a crystalline solid in which no solvent molecules occupy a specific crystallographic site.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “treating” with respect to the treatment of a disease-state in a patient include (i) inhibiting or ameliorating the disease-state in a patient, e.g., arresting or slowing its development; or (ii) relieving the disease-state in a patient, i.e., causing regression or cure of the disease-state. In the case of HIV, treatment includes reducing the level of HIV viral load in a patient.

The term “antiviral agent” as used herein is intended to mean an agent that is effective to inhibit the formation and/or replication of a virus in a human, including but not limited to agents that interfere with either host or viral mechanisms necessary for the formation and/or replication of a virus in a human. The term “antiviral agent” includes, for example, an HIV integrase catalytic site inhibitor selected from the group consisting: raltegravir (ISENTRESS®; Merck); elvitegravir (Gilead); soltegravir (GSK; ViiV); GSK 1265744 (GSK; ViiV) and dolutegravir; an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSK/ViiV); and lersivirine (Pfizer/ViiV); an HIV protease inhibitor selected from the group consisting of: atazanavir (REYATAZ® BMS); darunavir (PREZISTA®; J&J); indinavir (CRIXIVAN®; Merck); lopinavir (KELETRA®; Abbott); nelfinavir (VIRACEPT®; Pfizer); saquinavir (INVIRASE®; Hoffmann-LaRoche); tipranavir (APTIVUS®; BI); ritonavir (NORVIR®; Abbott); and fosamprenavir (LEXIVA®; GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Mynad Genetics).

As used herein, the term “tabletability” refers to the capacity of a powdered material to be transformed into a tablet of specified strength under the effect of compaction pressure. Tabletability is the tensile strength as a function of compression force and describes the effectiveness of the applied pressure in increasing the tensile strength of the tablet.

### Hemi-Succinate Salt of Compound (I)

The hemi-succinate salt of Compound (I) can be isolated in a non-crystalline form, a crystalline form or a mixture of both. The non-crystalline or crystalline forms may exist as a solvate or non-solvate.

### Form A

Crystalline Form A of the hemi-succinate salt of Compound (I) exhibits minimal weight loss during heating up to 175° C.

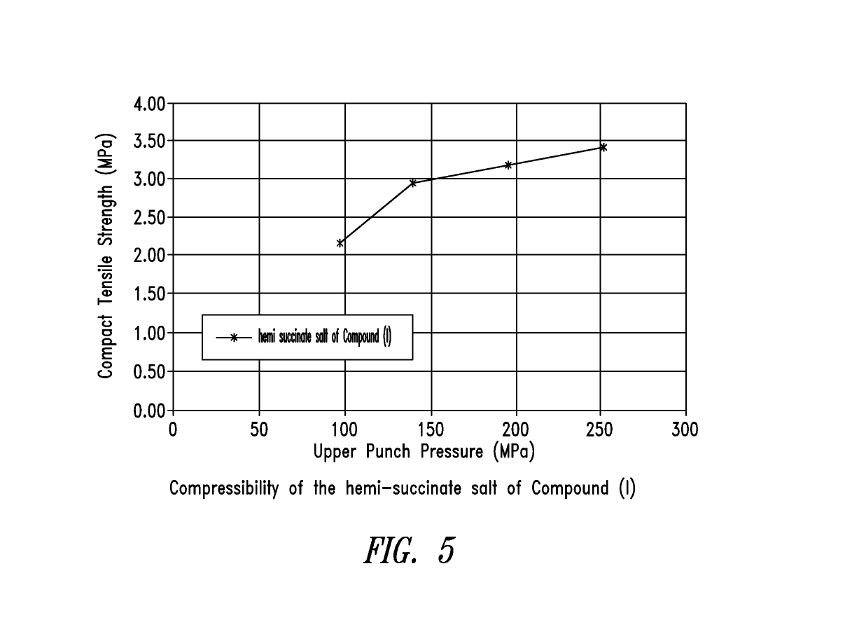

In addition, the hemi-succinate salt of Compound (II), Form A, advantageously exhibits improved manufacturability and stability. In particular, the hemi-succinate salt of Compound (I), Form A, has improved tabletability, thus allowing for the development of tablets including Compound (I) as either a single agent or in combination with other active pharmaceutical ingredients (APIs) as a single tablet regimen. FIG. 5 shows the tabletability of an immediate release formulation of the hemi-succinate salt of Compound (I), Form A. Table 1 below shows the tablet tensile strength values as a function of upper punch compression pressure as depicted in FIG. 5.

TABLE 1
Tabletability of the hemi-succinate salt of Compound (I)

| Upper Punch Compression | Tablet Tensile Strength |
|---|---|
| Pressure (MPa) | (MPa) |
| 98 | 2.17 |
| 140 | 2.94 |
| 197 | 3.18 |
| 253 | 3.41 |

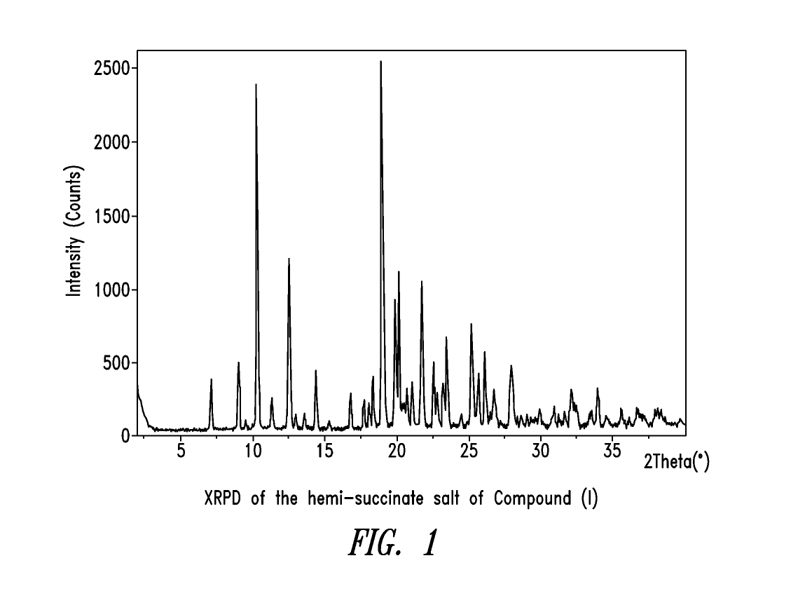

The XRPD pattern of the hemi-succinate salt of Compound (I), Form A, is shown in FIG. 1. A list of peak positions and relative intensities for the XRPD pattern in Certain characteristic peak positions and relative intensities for the XRPD pattern in FIG. 1 for the hemi-succinate salt of Compound (I), Form A, are shown in the following Table 2.

TABLE 2
Hemi-succinate salt of Compound (I)

| Angle | |
|---|---|
| 2-Theta ° | Relative Intensity % |
| 7.1 | 15 |
| 9.0 | 18 |
| 10.3 | 100 |
| 12.5 | 47 |
| 18.9 | 93 |
| 20.1 | 46 |
| 22.8 | 10 |
| 25.1 | 30 |
| 26.1 | 21 |
| 29.9 | 4 |

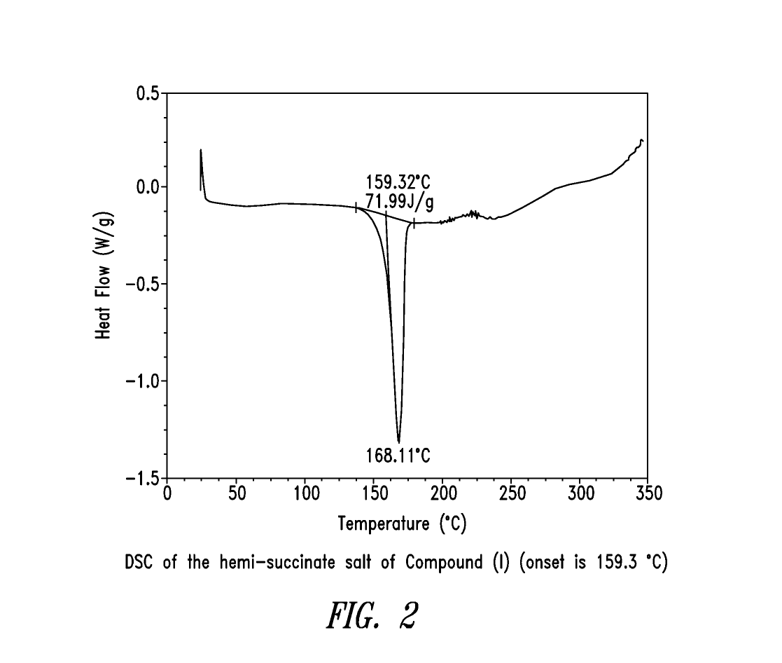

FIG. 2 shows the DSC thermogram for the hemi-succinate salt of Compound (I), Form A, crystals where the DSC is performed at a heating rate of 10° C. per minute in a crimped cup.

One embodiment of the invention is directed to a crystalline hemi-succinate salt of Compound (I), Form A, having an X-ray powder diffraction pattern (XRPD) including peaks at 7.1, 10.3 and 12.5 degrees 2θ (±0.2 degrees 2θ) when measured using CuKα radiation.

A further embodiment is directed to a crystalline hemi-succinate salt of Compound (I), Form A, having an XRPD pattern including peaks at 7.1, 10.3 and 12.5 degrees 2θ (±0.2 degrees 2θ) as described above and further including peaks at 18.9, 20.1 and 25.1 degrees 2θ (±0.2 degrees 2θ) when measured using CuKα radiation.

A further embodiment is directed to a crystalline hemi-succinate salt of Compound (I), Form A, having an XRPD pattern including peaks at 7.1, 10.3, 12.5, 18.9, 20.1 and 25.1 degrees 2θ (±0.2 degrees 2θ) as described above and further including peaks at 9.0, 22.8, 26.1 and 29.9 degrees 2θ (±0.2 degrees 2θ) when measured using CuKα radiation.

A further embodiment is directed to a crystalline hemi-succinate salt of Compound (I), Form A, exhibiting an XRPD pattern substantially the same as that shown in FIG. 1.

A further embodiment is directed to a crystalline hemi-succinate salt of Compound (I), Form A, having a DSC thermogram substantially the same as that shown in FIG. 2.

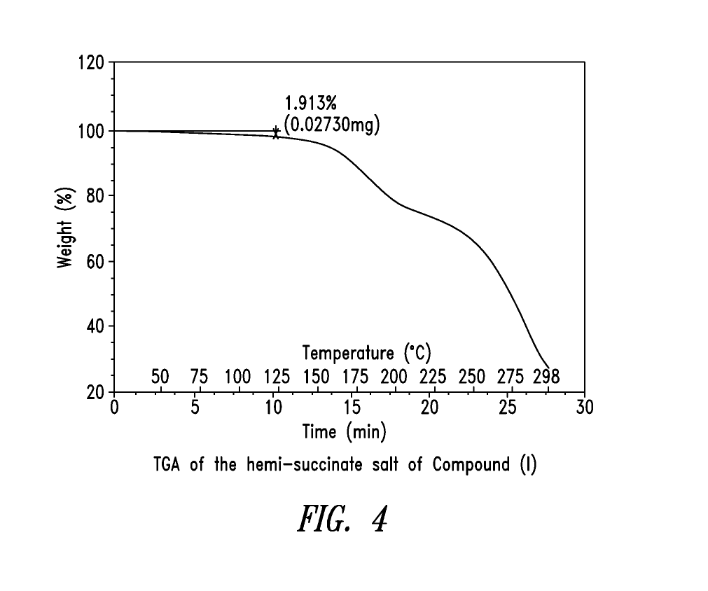

A further embodiment is directed to a crystalline hemi-succinate salt of Compound (I), Form A, having a TGA curve substantially the same as that shown in FIG. 4.

A further embodiment is directed to a crystalline hemi-succinate salt of Compound (I), Form A, having an XRPD pattern including peaks at 7.1, 10.3 and 12.5 degrees 2θ (±0.2 degrees 2θ) as described above and also exhibiting a DSC thermogram substantially the same as that shown in FIG. 2.

A further embodiment is directed to a crystalline hemi-succinate salt of Compound (I), Form A, having an XRPD pattern including peaks at 7.1, 10.3 and 12.5 degrees 2θ (±0.2 degrees 2θ) as described above and also exhibiting a TGA curve substantially the same as that shown in FIG. 4.

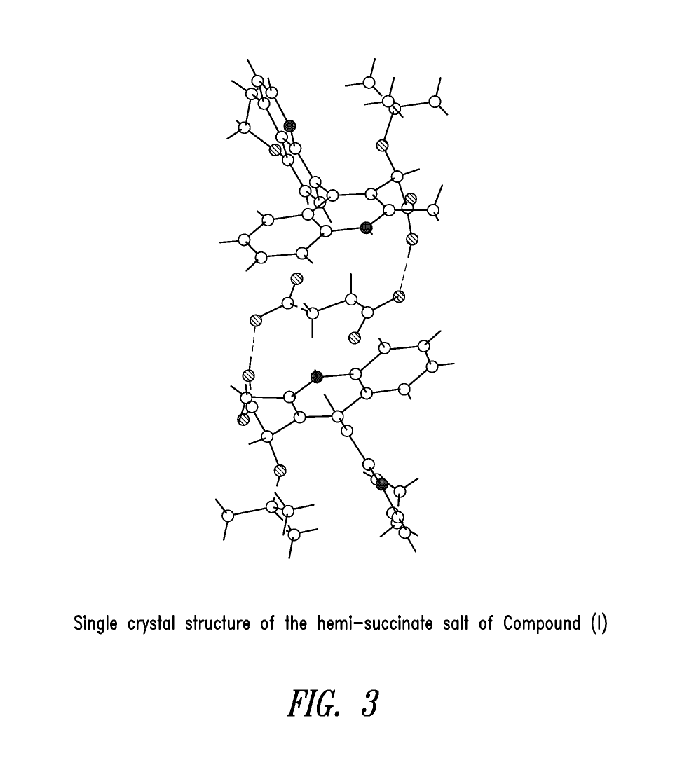

The single crystal structure of the hemi-succinate salt of Compound (I), Form A, is shown in FIG. 3.

Other alternative embodiments are directed to a quantity of a hemi-succinate salt of Compound (I) wherein at least about 50%, at least about 75%, at least about 95%, at least about 99%, or about 100%, of said substance is present in crystalline Form A as characterized by any of the abovementioned XRPD spectra defined embodiments. The presence of such amounts of hemi-succinate salt of Compound (I), Form A, is typically measurable using XRPD analysis of the compound.

Additional embodiments are directed to a pharmaceutical composition including a hemi-succinate salt of Compound (I) and a pharmaceutically acceptable carrier or diluent, wherein at least about 50%, at least about 75%, at least about 95%, at least about 99%, or about 100%, of said hemi-succinate salt of Compound (I), Form A, in the composition is present in crystalline form as characterized by any of the abovementioned XRPD spectrum defined embodiments.

Still further embodiments are directed to a pharmaceutical composition including a hemi-succinate salt of Compound (I) and a pharmaceutically acceptable carrier or diluent and further including at least one other antiviral agent, wherein at least about 50%, at least about 75%, at least about 95%, at least about 99%, or about 100%, of said hemi-succinate salt of Compound (I), Form A, in the composition is present in crystalline Form A as characterized by any of the abovementioned XRPD spectrum defined embodiments.

The present invention provides a process for the preparation of a crystalline form of Compound (I), Form A, which includes crystallizing a hemi-succinate salt of Compound (I) from a solution in solvents under conditions which yield the crystalline form of Compound (I), Form A. The precise conditions under which the crystalline form of Compound (I), Form A, is formed may be empirically determined and it is only possible to give methods which have been found to be suitable in practice. As one of skill in the art will appreciate, in each of the following synthetic processes, the recited steps may (i) occur individually or one or more steps may combined into a single step, (ii) occur in the order recited or in an alternative order and (iii) occur optionally.

It has been found that the hemi-succinate salt of Compound (I), Form A, may be prepared by a process including the following steps, which process is also an embodiment of the present invention:
(i) dissolving Compound (I) in a suitable solvent to obtain a mixture;
(ii) slowly heating the mixture of step (i) with stirring to a temperature to obtain a solution or slurry;
(iii) slowly adding succinic acid to the solution or slurry of step (ii) to obtain a solution;
(iv) optionally slowly adding a solution of sodium hydroxide to the solution of step (iii);
(v) optionally slowly cooling the solution of the preceding step;
(vi) optionally adding a seed including a hemi-succinate salt of Compound (I) to the solution of the preceding step to obtain a slurry;
(vii) optionally slowly adding a solution of sodium hydroxide to the solution or slurry of the preceding step;
(viii) cooling the mixture of the preceding step; and
(ix) collecting the solid material obtained in step (viii) to obtain the hemi-succinate salt of Compound (I).

In step (i), an exemplary suitable solvent includes an aliphatic alcohol, for example, isopropanol, water, or a combination thereof. In certain embodiments, the suitable solvent of step (i) includes a mixture of isopropanol and water.

In step (iii), succinic acid can advantageously be added in excess, thereby enabling substantially complete formation of the hemi-succinate salt of Compound (I).

In step (iv), in certain embodiments, the sodium hydroxide is in an aqueous solution.

In step (vii), in certain embodiments, the sodium hydroxide is in an aqueous solution.

The resulting crystals of the hemi-succinate form of Compound (I), Form A, are recovered by any conventional methods known in the art.

In the final step (ix), the resulting solids obtained in step (viii) are collected and dried at high temperature using conventional collection and high-temperature drying techniques, for example, filtration and vacuum oven.

### Pharmaceutical Compositions and Methods

The aforementioned hemi-succinate salt of Compound (I) and crystalline Form A of the hemi-succinate salt of Compound (I) are useful as anti-HIV agents in view of the demonstrated inhibitory activity of Compound (I) against HIV integrase. These forms are therefore useful in treatment of HIV infection in a human and can be used for the preparation of a pharmaceutical composition for treating an HIV infection or alleviating one or more symptoms thereof in a patient. The appropriate dosage amounts and regimens for a particular patient can be determined by methods known in the art and by reference to the disclosure in WO 2007/131350 and WO 2009/062285. Generally, a therapeutically effective amount for the treatment of HIV infection in the human is administered. In one embodiment, about 50 mg to 1000 mg, more preferably from about 50 mg to about 400 mg, is administered per adult human per day in single or multiple doses.

Specific optimal dosage and treatment regimens for any particular patient will of course depend upon a variety of factors, including the age, body weight, general health status, sex, diet, time of administration, rate of excretion, drug combination, the severity and course of the infection, the patient's disposition to the infection and the judgment of the treating physician. In general, the compound is most desirably administered at a concentration level that will generally afford antivirally effective results without causing any harmful or deleterious side effects.

The hemi-succinate salt of Compound (I) or crystalline Form A thereof at a selected dosage level is typically administered to the patient via a pharmaceutical composition. See, e.g., the description in WO 2007/131350 and WO 2009/062285 for the various types of compositions that may be employed in the present invention. The pharmaceutical composition may be administered orally, parenterally or via an implanted reservoir. The term parenteral as used herein includes subcutaneous, intracutaneous, intravenous, intramuscular, intra-articular, intrasynovial, intrasternal, intrathecal, and intralesional injection or infusion techniques. In certain specific embodiments, the hemi-succinate salt of Compound (I) or crystalline Form A thereof is administered orally or by injection.

In some embodiments, the pharmaceutical compositions of this invention contain any conventional non-toxic pharmaceutically-acceptable carriers, diluents, adjuvants, excipients or vehicles. In some embodiments, the pH of the formulation is adjusted with pharmaceutically acceptable acids, bases or buffers to enhance the stability of the formulated compound or its delivery form.

In one embodiment, the pharmaceutical composition is in the form of a sterile injectable preparation, for example, as a sterile injectable aqueous or oleaginous suspension. This suspension is formulated according to techniques known in the art using suitable dispersing or wetting agents (such as, for example, Tween 80) and suspending agents.

In certain embodiments, the pharmaceutical compositions is in the form of separate oral pharmaceutical compositions including the hemi-succinate salt of Compound (I) or crystalline Form A of the hemi-succinate salt of Compound (I) and at least one pharmaceutically acceptable carrier or diluent. In some embodiments, the pharmaceutical compositions are in the form of separate oral pharmaceutical compositions including the hemi-succinate salt of Compound (I) or crystalline Form A of the hemi-succinate salt of Compound (I), and one or more further antiviral agent. Exemplary orally acceptable dosage forms for the oral pharmaceutical compositions include, but are not limited to, tablets, capsules (e.g., hard or soft gelatin capsules), including liquid-filled capsules, and aqueous suspensions and solutions. In the case of tablets for oral use, carriers which are commonly used include lactose, microcrystalline cellulose and corn starch. Lubricating agents, such as magnesium stearate, are also typically added. For oral administration in a capsule form, useful diluents include lactose, microcrystalline cellulose and dried corn starch. Examples of soft gelatin capsules that can be used include those disclosed in U.S. Pat. No. 5,985,321. When aqueous suspensions are administered orally, the active ingredient is combined with emulsifying and suspending agents. If desired, certain sweetening and/or flavoring and/or coloring agents may be added.

Other suitable vehicles or carriers for the above noted formulations and compositions can be found in standard pharmaceutical texts. e.g., in “Remington's Pharmaceutical Sciences”, 19^{th} ed., Mack Publishing Company, Easton, Pa., 1995.

Certainly, when the crystalline Form A hemi-succinate salt of Compound (I) is formulated in a liquid vehicle, for example, as a liquid solution or suspension for oral administration or by injection, including for example in liquid-filled capsules, the crystalline Form A hemi-succinate salt of Compound (I) loses its crystalline nature. Nevertheless, the final liquid-based pharmaceutical composition contains the novel hemi-succinate salt of Compound (I) and it is therefore to be considered a separate embodiment embraced by the present invention. It was only by discovering a method for preparing the hemi-succinate salt in a stable crystalline form that the present inventors enabled efficient pharmaceutical processing and pharmaceutical formulation manufacture using the hemi-succinate salt form. Therefore, the final pharmaceutical formulation containing the hemi-succinate salt form which was thereby enabled by this discovery is considered another aspect and embodiment of the present invention.

### Methods of Characterization

### X-Ray Powder Diffraction

X-ray powder diffraction analyses were conducted on a PANalytical X'Pert-Pro X-Ray Powder Diffractometer, available from PANalytical of The Netherlands, using CuKα radiation (1.54 Å). The tube power was set to 45 kV and 40 mA. Step scans were run from 2 to 40° 2θ, at 0.017° per step, 15.875 sec per step. Samples were prepared for analysis by filling a zero background silicon holder.

### DSC Analysis

The DSC analysis was conducted on a TA instruments DSC Q 2000. The differential scanning calorimetry curve was obtained on a sample heated at 10° C. per minute in a crimped cup under a nitrogen flow.

### TGA

The TGA analysis was conducted on a TA instruments DSC Q 2000 IR. The thermal gravimetric curve was obtained on a sample heated at 10° C. per minute in a open cup under a nitrogen flow.

In order that this invention to be more fully understood, the following examples are set forth. These examples are for the purpose of illustrating embodiments of this invention, and are not to be construed as limiting the scope of the invention in any way. The reactants used in the examples below may be obtained either as described herein, or if not described herein, are themselves either commercially available or may be prepared from commercially available materials by methods known in the art. Certain starting materials, for example, may be obtained by methods described in the International Patent Applications WO 2007/131350 and WO 2009/062285.

Unless otherwise specified, solvents, temperatures, pressures, and other reaction conditions may be readily selected by one of ordinary skill in the art. Typically, reaction progress may be monitored by High Pressure Liquid Chromatography (HPLC), if desired, and intermediates and products may be purified by chromatography on silica gel and/or by recrystallization.

### EXAMPLES

Abbreviations or symbols used herein include:

Ac: acetyl; AcOH: acetic acid; Ac₂O: acetic anhydride; Bu: butyl; DMAc: N,N-Dimethylacetamide; ee: enantiomeric excess; Eq: equivalent; Et: ethyl; EtOAc: ethyl acetate; EtOH: ethanol; GC: gas chromatography; HPLC: high performance liquid chromatography; IPA: isopropyl alcohol; ⁱPr or i-Pr: 1-methylethyl (iso-propyl); KF: Karl Fischer; LOD: limit of detection; Me: methyl; MeCN: acetonitrile; MeOH: methanol; MS: mass spectrometry (ES: electrospray); MTBE: methyl-t-butyl ether; BuLi: n-butyl lithium; NMR: nuclear magnetic resonance spectroscopy; Pr: propyl; tert-butyl or t-butyl: 1,1-dimethylethyl; TFA: trifluoroacetic acid and; THF: tetrahydrofuran.

### Example 1

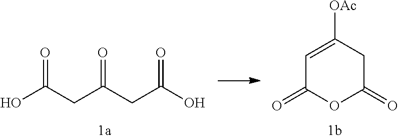 [SMILES: O=C(O)CC(=O)CC(=O)O ; CC(=O)OC1=CC(=O)OC(=O)C1]

1a (600 g, 4.1 mol) was charged into a dry reactor under nitrogen followed by addition of Ac₂O (1257.5 g, 12.3 mol, 3 eq.). The resulting mixture was heated at 40° C. at least for 2 hours. The batch was then cooled to 30° C. over 30 minutes. A suspension of 1b in toluene was added to seed the batch if no solid was observed. After toluene (600 mL) was added over 30 minutes, the batch was cooled to −5 to −10° C. and was held at this temperature for at least 30 minutes. The solid was collected by filtration under nitrogen and rinsed with heptanes (1200 mL). After being dried under vacuum at room temperature, the solid was stored under nitrogen at least below 20° C. The product 1b was obtained with 77% yield. ¹H NMR (500 MHz, CDCl₃): δ=6.36 (s, 1H), 3.68 (s, 2H), 2.30 (s, 3H).

### Example 2

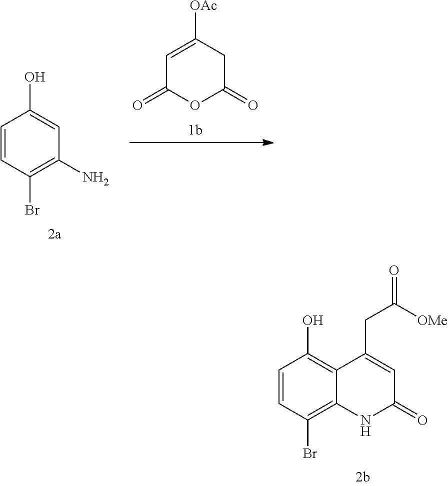 [SMILES: Nc1cc(O)ccc1Br ; CC(=O)OC1=CC(=O)OC(=O)C1 ; COC(=O)Cc1cc(=O)[nH]c2c(Br)ccc(O)c12]

2a (100 g, 531 mmol) and 1b (95 g, 558 mmol) were charged into a clean and dry reactor under nitrogen followed by addition of fluorobenzene (1000 mL). After being heated at 35-37° C. for 4 hours, the batch was cooled to 23° C. Concentrated H₂SO₄ (260.82 g, 2659.3 mmol, 5 eq.) was added while maintaining the batch temperature below 35° C. The batch was first heated at 30-35° C. for 30 minutes and then at 40-45° C. for 2 hours. 4-Methyl morpholine (215.19 g, 2127 mmol, 4 eq.) was added to the batch while maintaining the temperature below 50° C. Then the batch was agitated for 30 minutes at 40-50° C. MeOH (100 mL) was then added while maintaining the temperature below 55° C. After the batch was held at 50-55° C. for 2 hours, another portion of MeOH (100 mL) was added. The batch was agitated for another 2 hours at 50-55° C. After fluorobenzene was distilled to a minimum amount, water (1000 mL) was added. Further distillation was performed to remove any remaining fluorobenzene. After the batch was cooled to 30° C., the solid was collected by filtration with cloth and rinsed with water (400 mL) and heptane (200 mL). The solid was dried under vacuum below 50° C. to reach KF<0.1%. Typically, the product 2b was obtained in 90% yield with 98 wt %. ¹H NMR (500 MHz, DMSO-d₆): δ=10.83 (s, 1H), 9.85 (s, bs, 1H), 7.6 (d, 1H, J=8.7 Hz), 6.55 (d, 1H, J=8.7 Hz), 6.40 (s, 1H), 4.00 (s, 2H), 3.61 (s, 3H).

### Example 3

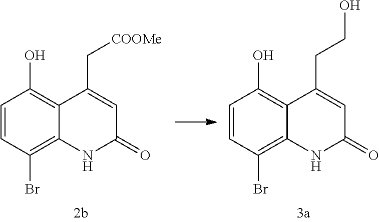 [SMILES: COC(=O)Cc1cc(=O)[nH]c2c(Br)ccc(O)c12 ; O=c1cc(CCO)c2c(O)ccc(Br)c2[nH]1]

2b (20 g, 64 mmol) was charged into a clean and dry reactor followed by addition of THF (140 mL). After the resulting mixture was cooled to 0° C., Vitride® (Red-Al, 47.84 g, 65 wt %, 154 mmol) in toluene was added while maintaining an internal temperature at 0-5° C. After the batch was agitated at 5-10° C. for 4 hours, IPA (9.24 g, 153.8 mmol) was added while maintaining the temperature below 10° C. Then the batch was agitated at least for 30 minutes below 25° C. A solution of HCl in IPA (84.73 g, 5.5 M, 512 mmol) was added into the reactor while maintaining the temperature below 40° C. After about 160 mL of the solvent was distilled under vacuum below 40° C., the batch was cooled to 20-25° C. and then aqueous 6M HCl (60 mL) was added while maintaining the temperature below 40° C. The batch was cooled to 25° C. and agitated for at least 30 minutes. The solid was collected by filtration, washed with 40 mL of IPA and water (1V/1V), 40 mL of water and 40 mL of heptanes. The solid was dried below 60° C. in a vacuum oven to reach KF<0.5%. Typically, the product 3a was obtained in 90-95% yield with 95 wt %. ¹H NMR (400 MHz, DMSO-d₆): δ=10.7 (s, 1H), 9.68 (s, 1H), 7.59 (d, 1H, J=8.7 Hz), 6.64 (, 1H, J=8.7 Hz), 6.27 (s, 1H), 4.62 (bs, 1H), 3.69 (t, 2H, J=6.3 Hz), 3.21 (t, 2H, J=6.3 Hz).

### Example 4

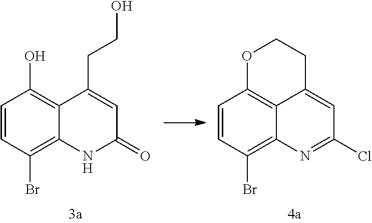 [SMILES: O=c1cc(CCO)c2c(O)ccc(Br)c2[nH]1 ; Clc1cc2c3c(ccc(Br)c3n1)OCC2]

3a (50 g, 174.756 mmol) and acetonitrile (200 mL) were charged into a dry and clean reactor. After the resulting mixture was heated to 65° C., POCl₃ (107.18 g, 699 mmol, 4 eq.) was added while maintaining the internal temperature below 75° C. The batch was then heated at 70-75° C. for 5-6 h. The batch was cooled to 20° C. Water (400 mL) was added at least over 30 minutes while maintaining the internal temperature below 50° C. After the batch was cooled to 20-25° C. over 30 minutes, the solid was collected by filtration and washed with water (100 mL). The wet cake was charged back into the reactor followed by addition of 1M NaOH (150 mL). After the batch was agitated at least for 30 minutes at 25-35° C., verify that the pH was greater than 12. Otherwise, more 6M NaOH was needed to adjust the pH>12. After the batch was agitated for 30 minutes at 25-35° C., the solid was collected by filtration, washed with water (200 mL) and heptanes (200 mL). The solid was dried in a vacuum oven below 50° C. to reach KF<2%. Typically, the product 4a was obtained at about 75-80% yield. ¹H NMR (400 MHz, CDCl₃): δ=7.90 (d, 1H, J=8.4 Hz), 7.16 (s, 1H), 6.89 (d, 1H, J=8.4 Hz), 4.44 (t, 2H, J=5.9 Hz), 3.23 (t, 2H, J=5.9 Hz). ¹³C NMR (100 MHz, CDCl₃): δ=152.9, 151.9, 144.9, 144.1, 134.6, 119.1, 117.0, 113.3, 111.9, 65.6, 28.3.

### Example 5

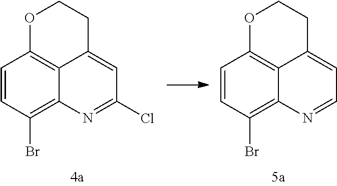 [SMILES: Clc1cc2c3c(ccc(Br)c3n1)OCC2 ; Brc1ccc2c3c(ccnc13)CCO2]

Zn powder (54 g, 825 mmol, 2.5 eq.) and TFA (100 mL) were charged into a dry and clean reactor. The resulting mixture was heated to 60-65° C. A suspension of 4a (100 g, 330 mmol) in 150 mL of TFA was added to the reactor while maintaining the temperature below 70° C. The charge line was rinsed with TFA (50 mL) into the reactor. After 1 hour at 65±5° C., the batch was cooled to 25-30° C. Zn powder was filtered off by passing the batch through a Celite pad and washing with methanol (200 mL). About 400 mL of solvent was distilled off under vacuum. After the batch was cooled to 20-25° C., 20% NaOAc (ca. 300 mL) was added at least over 30 minutes to reach pH 5-6. The solid was collected by filtration, washed with water (200 mL) and heptane (200 mL), and dried under vacuum below 45° C. to reach KF≦2%. The solid was charged into a dry reactor followed by addition of loose carbon (10 wt %) and toluene (1000 mL). The batch was heated at least for 30 minutes at 45-50° C. The carbon was filtered off above 35° C. and rinsed with toluene (200 mL). The filtrate was charged into a clean and dry reactor. After about 1000 mL of toluene was distilled off under vacuum below 50° C., 1000 mL of heptane was added over 30 minutes at 40-50° C. Then the batch was cooled to 0±5° C. over 30 minutes. After 30 minutes, the solid was collected and rinsed with 200 mL of heptane. The solid was dried under vacuum below 45° C. to reach KF≦500 ppm. Typically, the product 5a was obtained in about 90-95% yield. ¹H NMR (400 MHz, CDCl₃): δ=8.93 (m, 1H), 7.91 (dd, 1H, J=1.5, 8 Hz), 7.17 (m 1H), 6.90 (dd, 1H, J=1.6, 8.0 Hz), 4.46-4.43 (m, 2H), 3.28-3.23 (m, 2H). ¹³C NMR (100 MHz, CDCl₃): δ=152.8, 151.2, 145.1, 141.0, 133.3, 118.5, 118.2, 114.5, 111.1, 65.8, 28.4.

### Example 6

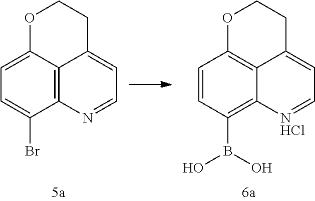

5a (1.04 kg, 4.16 mol) and toluene (8 L) were charged into the reactor. The batch was agitated and cooled to −50 to −55° C. nBuLi solution (2.5 M in hexanes, 1.69 L, 4.23 mol) was charged slowly while maintaining the internal temperature between −45 to −50° C. The batch was agitated at −45° C. for 1 hour after addition. A solution of triisopropyl borate (0.85 kg, 4.5 mol) in MTBE (1.48 kg) was charged. The batch was warmed to 10° C. over 30 minutes. A solution of 5 N HCl in IPA (1.54 L) was charged slowly at 10° C., and the batch was warmed to 20° C. and stirred for 30 minutes. It was seeded with 6a crystal (10 g). A solution of aqueous concentrated HCl (0.16 L) in IPA (0.16 L) was charged slowly at 20° C. in three portions at 20 minute intervals, and the batch was agitated for 1 hour at 20° C. The solid was collected by filtration, rinsed with MTBE (1 kg), and dried to provide 6a (943 g, 88.7% purity, 80% yield). ¹H NMR (400 MHz, D₂O): δ 8.84 (d, 1H, J=4 Hz), 8.10 (m, 1H), 7.68 (d, 1H, J=6 Hz), 7.09 (m, 1H), 4.52 (m, 2H), 3.47 (m, 2H).

### Example 7

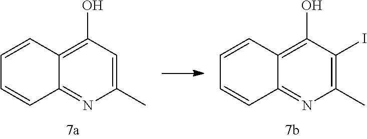

Iodine stock solution was prepared by mixing iodine (57.4 g, 0.23 mol) and sodium iodide (73.4 g, 0.49 mol) in water (270 mL). Sodium hydroxide (28.6 g, 0.715 mol) was charged into 220 mL of water. 4-Hydroxy-2 methylquinoline 7a (30 g, 0.19 mol) was charged, followed by acetonitrile (250 mL). The mixture was cooled to 10° C. with agitation. The above iodine stock solution was charged slowly over 30 minutes. The reaction was quenched by addition of sodium bisulfite (6.0 g) in water (60 mL). Acetic acid (23 mL) was charged over a period of 1 hour to adjust the pH of the reaction mixture between 6 and 7. The product was collected by filtration, washed with water and acetonitrile, and dried to give 7b (53 g, 98%). MS 286 [M+1].

### Example 8

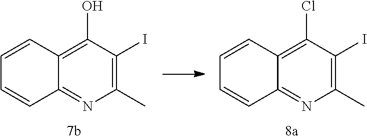

4-Hydroxy-3-iodo-2-methylquinoline 7b (25 g, 0.09 mol) was charged to a 1-L reactor. Ethyl acetate (250 mL) was charged, followed by triethylamine (2.45 mL, 0.02 mol) and phosphorus oxychloride (12 mL, 0.13 mol). The reaction mixture was heated to reflux until complete conversion (˜1 hour), then the mixture was cooled to 22° C. A solution of sodium carbonate (31.6 g, 0.3 mol) in water (500 mL) was charged. The mixture was stirred for 20 minutes. The aqueous layer was extracted with ethyl acetate (120 mL). The organic layers were combined and concentrated under vacuum to dryness. Acetone (50 mL) was charged. The solution was heated to 60° C. Water (100 mL) was charged, and the mixture was cooled to 22° C. The product was collected by filtration and dried to give 8a (25 g, 97.3% pure, 91.4% yield). MS 304 [M+1].

(Note: 8a is a known compound with CAS #1033931-93-9. See references: (a) J. Org Chem. 2008, 73, 4644-4649. (b) Molecules 2010, 15, 3171-3178. (c) Indian J. Chem. Sec B: Org. Chem. Including Med Chem. 2009, 48B(5), 692-696.)

### Example 9

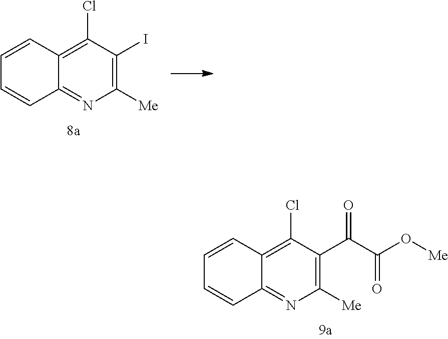 [SMILES: Cc1nc2ccccc2c(Cl)c1I ; COC(=O)C(=O)c1c(C)nc2ccccc2c1Cl]

8a (100 g, 0.33 mol) was charged to the reactor, followed by copper (I) bromide dimethyl sulfide complex (3.4 g, 0.017 mol) and dry THF (450 mL). The batch was cooled to −15 to −12° C. i-PrMgCl (2.0 M in THF, 173 mL, 0.346 mol) was charged into the reactor at the rate which maintains the batch temperature <−10° C. In a 2nd reactor, methyl chlorooxoacetate (33 mL, 0.36 mol) and dry THF (150 mL) was charged. The solution was cooled to −15 to −10° C. The content of the 1st reactor (Grignard/cuprate) was charged into the 2nd reactor at the rate which maintained the batch temperature <−10° C. The batch was agitated for 30 minutes at −10° C. Aqueous ammonium chloride solution (10%, 300 mL) was charged. The batch was agitated at 20-25° C. for 20 minutes and allowed to settle for 20 minutes. The aqueous layer was separated. Aqueous ammonium chloride solution (10%, 90 mL) and sodium carbonate solution (10%, 135 mL) were charged to the reactor. The batch was agitated at 20-25° C. for 20 minutes and allowed to settle for 20 minutes. The aqueous layer was separated. Brine (10%, 240 mL) was charged to the reactor. The batch was agitated at 20-25° C. for 20 minutes. The aqueous layer was separated. The batch was concentrated under vacuum to ˜¼ of the volume (about 80 mL left). 2-Propanol was charged (300 mL). The batch was concentrated under vacuum to ˜⅓ of the volume (about 140 mL left), and heated to 50° C. Water (70 mL) was charged. The batch was cooled to 20-25° C., stirred for 2 hours, cooled to −10° C. and stirred for another 2 hours. The solid was collected by filtration, washed with cold 2-propanol and water to provide 58.9 g of 9a obtained after drying (67.8% yield). ¹H NMR (400 MHz, CDCl₃): δ 8.08 (d, 1H, J=12 Hz), 7.97 (d, 1H, J=12 Hz), 7.13 (t, 1H, J=8 Hz), 7.55 (t, 1H, J=8 Hz), 3.92 (s, 3H), 2.63 (s, 3H). ¹³C NMR (100 MHz, CDCl₃): δ 186.6, 161.1, 155.3, 148.2, 140.9, 132.0, 129.0, 128.8, 127.8, 123.8, 123.7, 53.7, 23.6.

### Example 10

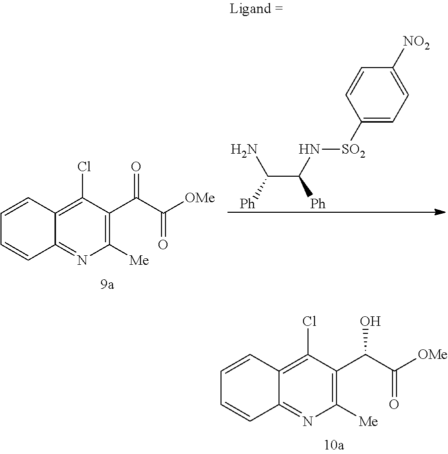 [SMILES: COC(=O)C(=O)c1c(C)nc2ccccc2c1Cl ; N[C@@H](c1ccccc1)[C@@H](NS(=O)(=O)c1ccc([N+](=O)[O-])cc1)c1ccccc1 ; COC(=O)[C@@H](O)c1c(C)nc2ccccc2c1Cl]

### Catalyst Preparation:

To a suitable sized, clean and dry reactor was charged dichloro(pentamethylcyclopentadienyl)rhodium(III) dimer (800 ppm relative to 9a, 188.5 mg) and the ligand (2000 ppm relative to 9a, 306.1 mg). The system was purged with nitrogen and then 3 mL of acetonitrile and 0.3 mL of triethylamine was charged to the system. The resulting solution was agitated at RT for not less than 45 minutes and not more than 6 hours.

### Reaction:

To a suitable sized, clean and dry reactor was charged 9a (1.00 equiv, 100.0 g (99.5 wt %), 377.4 mmol). The reaction was purged with nitrogen. To the reactor was charged acetonitrile (ACS grade, 4 L/Kg of 9a, 400 mL) and triethylamine (2.50 equiv, 132.8 mL, 943 mmol). Agitation was initiated. The 9a solution was cooled to Tᵢₙₜ=−5 to 0° C. and then formic acid (3.00 equiv, 45.2 mL, 1132 mmol) was charged to the solution at a rate to maintain Tᵢₙₜ not more than 20° C. The batch temperature was then adjusted to Tᵢₙₜ=−5 to −0° C. Nitrogen was bubbled through the batch through a porous gas dispersion unit (Wilmad-LabGlass No. LG-8680-110, VWR catalog number 14202-962) until a fine stream of bubbles was obtained. To the stirring solution at Tᵢₙₜ=−5 to 0° C. was charged the prepared catalyst solution from the catalyst preparation above. The solution was agitated at Tᵢₙₜ=−5 to 0° C. with the bubbling of nitrogen through the batch until HPLC analysis of the batch indicated no less than 98 A % conversion (as recorded at 220 nm, 10-14 h). To the reactor was charged isopropylacetate (6.7 L/Kg of 9a, 670 mL). The batch temperature was adjusted to Tᵢₙₜ=18 to 23° C. To the solution was charged water (10 L/Kg of 9a, 1000 mL) and the batch was agitated at Tᵢₙₜ=18 to 23° C. for no less than 20 minutes. The agitation was decreased and or stopped and the layers were allowed to separate. The lighter colored aqueous layer was cut. To the solution was charged water (7.5 L/Kg of 9a, 750 mL) and the batch was agitated at Tᵢₙₜ=18 to 23° C. for no less than 20 minutes. The agitation was decreased and or stopped and the layers were allowed to separate. The lighter colored aqueous layer was cut. The batch was then reduced to 300 mL (3 L/Kg of 9a) via distillation while maintaining Tₑₓₜ no more than 65° C. The batch was cooled to Tᵢₙₜ=35 to 45° C. and the batch was seeded (10 mg). To the batch at Tᵢₙₜ=35 to 45° C. charged heptane (16.7 L/Kg of 9a, 1670 mL) over no less than 1.5 hours. Adjusted the batch temperature to Tᵢₙₜ=−2 to 3° C. over no less than 1 hour, and agitated the batch at Tᵢₙₜ=−2 to 3° C. for no less than 1 hour. Collected the solids by filtration. Used the filtrate to rinse the reactor (Filtrate is cooled to Tᵢₙₜ=−2 to 3° C. before filtration) and the solids were suction dried for no less than 2 hours. The solids were dried until the LOD was no more than 4% to obtain 82.7 g of 10a (99.6-100 wt %, 98.5% ee, 82.5% yield). ¹H-NMR (CDCl₃, 400 MHz) δ: 8.20 (d, J=8.4 Hz, 1H), 8.01 (d, J=8.4 Hz, 1H), 7.73 (t, J=7.4 Hz, 1H), 7.59 (t, J=7.7 Hz, 1H), 6.03 (s, 1H), 3.93 (s, 1H), 3.79 (s, 3H), 2.77 (s, 3H). ¹³C-NMR (CDCl₃, 100 MHz) δ: 173.5, 158.3, 147.5, 142.9, 130.7, 128.8, 127.7, 127.1, 125.1, 124.6, 69.2, 53.4, 24.0.

### Example 11

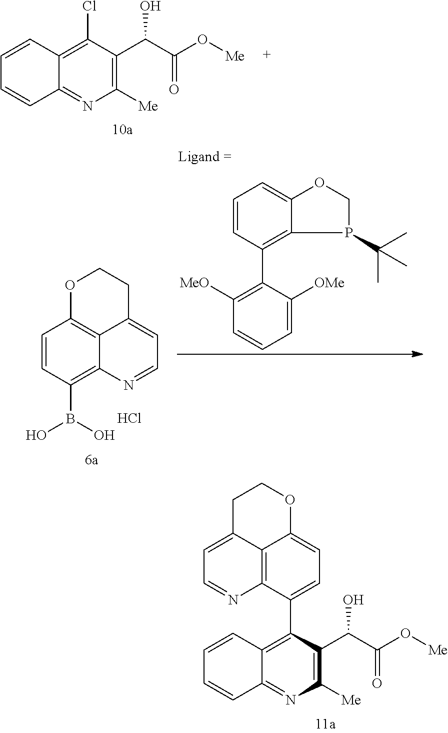

10a (2.45 kg, 96.8% purity, 8.9 mol), 6a (2.5 kg, 88.7% purity, 8.82 mol), tris(dibenzylideneacetone)dipalladium(0) (Pd₂ dba₃, 40 g, 0.044 mol), (S)-3-tert-butyl-4-(2,6-dimethoxyphenyl)-2,3-dihydrobenzo[d][1,3]oxaphosphole (32 g, 0.011 mol), sodium carbonate (1.12 kg, 10.58 mol), 1-pentanol (16.69 L), and water (8.35 L) were charged to the reactor. The mixture was de-gassed by sparging with argon for 10-15 minutes, was heated to 60-63° C., and was agitated until HPLC analysis of the reaction shows <1 A % (220 nm) of the 6a relative to the combined two atropisomer products (˜15 hours). The batch was cooled to 18-23° C. Water (5 L) and heptane (21 L) were charged. The slurry was agitated for 3-5 hours. The solids were collected by filtration, washed with water (4 L) and heptane/toluene mixed solvent (2.5 L toluene/5 L heptane), and dried. The solids were dissolved in methanol (25 L) and the resulting solution was heated to 50° C. and circulated through a CUNO carbon stack filter. The solution was distilled under vacuum to ˜5 L. Toluene (12 L) was charged. The mixture was distilled under vacuum to ˜5 L and cooled to 22° C. Heptane (13 L) was charged to the contents over 1 hour and the resulting slurry was agitated at 20-25° C. for 3-4 hours. The solids were collected by filtration and washed with heptanes to provide 2.58 kg of 11a obtained after drying (73% yield). ¹H NMR (400 MHz, CDCl₃): δ 8.63 (d, 1H, J=8 Hz), 8.03 (d, 1H, J=12 Hz), 7.56 (t, 1H, J=8 Hz), 7.41 (d, 1H, J=8 Hz), 7.19 (t, 1H, J=8 Hz), 7.09 (m, 2H), 7.04 (d, 1H, J=8 Hz), 5.38 (d, 1H, J=8 Hz), 5.14 (d, 1H, J=8 Hz), 4.50 (t, 2H, J=4 Hz), 3.40 (s, 3H), 3.25 (t, 2H, J=4 Hz), 2.91 (s, 3H). ¹³C NMR (100 MHz, CDCl₃): δ 173.6, 158.2, 154.0, 150.9, 147.3, 147.2, 145.7, 141.3, 132.9, 123.0, 129.4, 128.6, 127.8, 126.7, 126.4, 125.8, 118.1, 117.3, 109.9, 70.3, 65.8, 52.3, 28.5, 24.0.

### Example 12

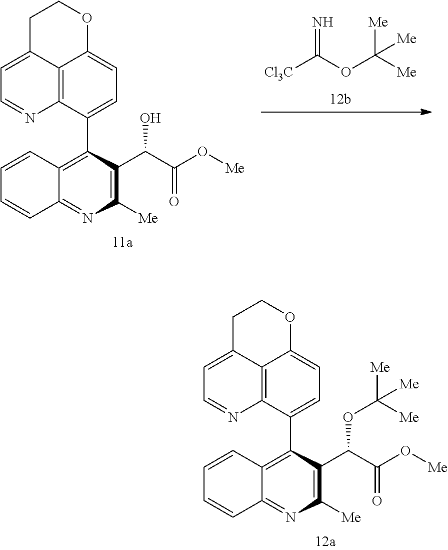

To a suitable clean and dry reactor under a nitrogen atmosphere was charged 11a (5.47 Kg, 93.4 wt %, 1.00 equiv, 12.8 mol) and fluorobenzene (10 vols, 51.1 kg) following by trifluoromethanesulfonimide (4 mol %, 143 g, 0.51 mol) as a 0.5 M solution in DCM (1.0 Kg). The batch temperature was adjusted to 35-41° C. and agitated to form a fine slurry. To the mixture was slowly charged t-butyl-2,2,2-trichloroacetimidate 12b as a 50 wt % solution (26.0 Kg of t-butyl-2,2,2-trichloroacetimidate (119.0 mol, 9.3 equiv), the reagent was ˜48-51 wt % with the remainder 52-49 wt % of the solution being ˜1.8:1 wt:wt heptane:fluorobenzene) over no less than 4 hours at Tᵢₙₜ=35-41° C. The batch was agitated at Tᵢₙₜ=35-41° C. until HPLC conversion (308 nm) was >96 A %, then cooled to Tᵢₙₜ=20-25° C. and then triethylamine (0.14 equiv, 181 g, 1.79 mol) was charged followed by heptane (12.9 Kg) over no less than 30 minutes. The batch was agitated at Tᵢₙₜ=20-25° C. for no less than 1 hour. The solids were collected by filtration. The reactor was rinsed with the filtrate to collect all solids. The collected solids in the filter were rinsed with heptane (11.7 Kg). The solids were charged into the reactor along with 54.1 Kg of DMAc and the batch temperature adjusted to Tᵢₙₜ=70-75° C. Water (11.2 Kg) was charged over no less than 30 minutes while the batch temperature was maintained at Tᵢₙₜ=65-75° C. 12a seed crystals (34 g) in water (680 g) was charged to the batch at Tᵢₙₜ=65-75° C. Additional water (46.0 Kg) was charged over no less than 2 hours while maintaining the batch temperature at Tᵢₙₜ=65-75° C. The batch temperature was adjusted to Tᵢₙₜ=18-25° C. over no less than 2 hours and agitated for no less than 1 hour. The solids were collected by filtration and the filtrate used to rinse the reactor. The solids were washed with water (30 Kg) and dried under vacuum at no more than 45° C. until the LOD<4% to obtain 12a (5.275 Kg, 99.9 A % at 220 nm, 99.9 wt % via HPLC wt % assay, 90.5% yield). ¹H-NMR (CDCl₃, 400 MHz) δ: 8.66-8.65 (m, 1H), 8.05 (d, J=8.3 Hz, 1H), 7.59 (t, J=7.3 Hz, 1H), 7.45 (d, J=7.8 Hz, 1H), 7.21 (t, J=7.6 Hz, 1H), 7.13-7.08 (m, 3H), 5.05 (s, 1H), 4.63-4.52 (m, 2H), 3.49 (s, 3H), 3.41-3.27 (m, 2H), 3.00 (s, 3H), 0.97 (s, 9H). ¹³C-NMR (CDCl₃, 100 MHz) δ: 172.1, 159.5, 153.5, 150.2, 147.4, 146.9, 145.4, 140.2, 131.1, 130.1, 128.9, 128.6, 128.0, 127.3, 126.7, 125.4, 117.7, 117.2, 109.4, 76.1, 71.6, 65.8, 51.9, 28.6, 28.0, 25.4.

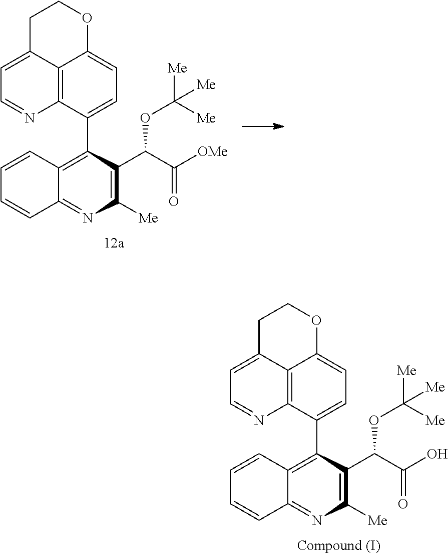

### Example 13

To a suitable clean and dry reactor under a nitrogen atmosphere was charged 12a (9.69 Kg, 21.2 mol) and ethanol (23.0 Kg). The mixture was agitated and the batch temperature was maintained at Tᵢₙₜ=20 to 25° C. 2 M sodium hydroxide (17.2 Kg) was charged at Tᵢₙₜ=20 to 25° C. and the batch temperature was adjusted to Tᵢₙₜ=60-65° C. over no less than 30 minutes. The batch was agitated at Tᵢₙₜ=60-65° C. for 2-3 hours until HPLC conversion was >99.5% area (12a is <0.5 area %). The batch temperature was adjusted to Tᵢₙₜ=50 to 55° C. and 2M aqueous HCl (14.54 Kg) was charged. The pH of the batch was adjusted to pH 5.0 to 5.5 (target pH 5.2 to 5.3) via the slow charge of 2M aqueous HCl (0.46 Kg) at Tᵢₙₜ=50 to 55° C. Acetonitrile was charged to the batch (4.46 Kg) at Tᵢₙₜ=50 to 55° C. A slurry of seed crystals (Compound (I), 20 g in 155 g of acetonitrile) was charged to the batch at Tᵢₙₜ=50 to 55° C. The batch was agitated at Tᵢₙₜ=50 to 55° C. for no less than 1 hour (1-2 hours). The contents were vacuum distilled to ˜3.4 vol (32 L) while maintaining the internal temperature at 45-55° C. A sample of the batch was removed and the ethanol content was determined by GC analysis; the criterion was no more than 10 wt % ethanol. If the ethanol wt % was over 10%, an additional 10% of the original volume was distilled and sampled for ethanol wt %. The batch temperature was adjusted to Tᵢₙₜ=18-22° C. over no less than 1 hour. The pH of the batch was verified to be pH=5-5.5 and the pH was adjusted, if necessary, with the slow addition of 2 M HCl or 2 M NaOH aqueous solutions. The batch was agitated at Tᵢₙₜ=18-22° C. for no less than 6 hours and the solids were collected by filtration. The filtrate/mother liquid was used to remove all solids from reactor. The cake with was washed with water (19.4 Kg) (water temperature was no more than 20° C.). The cake was dried under vacuum at no more than 60° C. for 12 hours or until the LOD was no more than 4% to obtain Compound (I) (9.52 Kg, 99.6 A % 220 nm, 97.6 wt % as determined by HPLC wt % assay, 99.0% yield).

### Example 14

### Hemi-Succinate Salt of Compound (I), Form A

Reactor A: Compound (I)

(23.0 g) was charged to a reactor followed by addition of isopropanol (150 mL) and water (1200 mL). The mixture was agitated and heated to 65° C. to form a slurry. Succinic acid (153.6 g) was then added to form a solution. To this solution was added a pre-made solution of sodium hydroxide (5.2 g) in water (20 mL) at 65° C. The resulting solution was cooled to 50° C. and then seeded with the hemi-succinate salt of Compound (I) (1.5 g). The resulting slurry was cooled to 45° C. and agitated for 2 hours.

Reactor B:

In a separate reactor, a sodium hydroxide (11.7 g) and water (450 mL) solution was prepared at 20° C. Compound (I) (135.0 g) was then added and agitated for ˜15 minutes to form a solution. This solution was slowly added to the slurry in Reactor A over 6 hours while maintaining the internal temperature of Reactor A batch at 45° C. After the addition was complete, the slurry was cooled to 20° C. The solids were collected by filtration, washed with water (450 mL) twice, and with a mixture of isopropanol/heptane (450 mL, 1/1 v/v). The resulting solids were dried in vacuum oven at 40° C. to yield the hemi-succinate salt of Compound (I), Form A, as a yellow solid (149.6 g, 92% yield). The product was assayed with 99.8% LCAP, 101 wt %, succinic acid content at 0.49 eq. The XRPD pattern was consistent with that of a standard of the hemi-succinate salt of Compound (I), Form A. ¹H NMR (400 MHz, DMSO-d₆) δ 12.26 (s, 2H), 8.51 (d, J=4.4 Hz, 1H), 7.94 (d, J=8 Hz, 1H), 7.62 (ddd, J=8.3, 6.8, 1.4 Hz, 1H), 7.44 (d, J=7.8 Hz, 1H), 7.29 (d, J=4.4 Hz, 1H), 7.25 (ddd, J=8.2, 6.8, 1.2 Hz, 1H), 7.11 (d, J=7.9 Hz, 1H), 6.93 (dd, J=8.4, 0.8 Hz, 1H), 4.99 (s, 1H), 4.63-4.35 (m, 2H), 3.50-3.12 (m, 2H), 2.85 (s, 3H), 2.40 (s, 2H), 0.82 (s, 9H).

### Example 15

### Single Crystal X-Ray Structure of the Hemi-Succinate Salt of Compound (I), Form A

The single crystal structure of the hemi-succinate salt of Compound (I), Form A, is shown in FIG. 3. The crystal structure solution was obtained by direct method, full-matrix least-squares refinement on F². The acquisition and structure is shown in Table 3 below.

TABLE 3
Crystal structure parameters for hemi-succinate salt of Compound (I)
Form A

| Empirical formula | C₂₇H₂₆N₂O₄•½(C₄H₆O₄) | |
|---|---|---|
| Formula weight | 501.54 | |
| Temperature | 90(2) K | |
| Wavelength | 1.54178 Å | |
| Crystal system | Tetragonal | |
| Space group | P4(3)2(1)2 | |
| Unit cell dimensions | a = 9.9969(8) Å | α = 90° |
| | b = 0.9969(8) Å | β = 90° |
| | c = 49.180(4) Å | γ = 90° |
| Volume | 4914.9(7) Å³ | |

| | |
|---|---|
| Z | 8 |
| Density (calculated) | 1.356 g/cm³ |
| Absorption coefficient | 0.781 mm⁻¹ |
| F(000) | 2120 |
| Theta range for data collection | 4.51 to 68.29° |
| Reflections collected | 17526 |
| Independent reflections | 4358 [R (int) = 0.0519] |
| Completeness to theta = 66.00° | 99.6% |

In addition, the single crystal structure results show in Table 3 and FIG. 3 indicate that the molar ratio of Compound (I), Form A, to succinic acid is 2:1, which confirms that this salt form of Compound (I) exists as a hemi-succinate salt.

Each reference, including all patents, patent applications, and publications cited in the present application is incorporated herein by reference in its entirety, as if each of them is individually incorporated. Further, it would be appreciated that, in the above teaching of invention, the skilled in the art could make certain changes or modifications to the invention, and these equivalents would still be within the scope of the invention defined by the appended claims of the application.

1. A hemi-succinate salt of Compound (I):

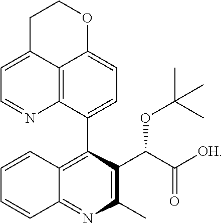

2. The hemi-succinate salt of Compound (I) according to claim 1 in crystalline Form A.

3. The crystalline hemi-succinate salt of Compound (I) according to claim 2 having an X-ray powder diffraction pattern comprising peaks at 7.1, 10.3 and 12.5 degrees 2θ (±0.2 degrees 2θ) when measured using CuKα radiation.

4. The crystalline hemi-succinate salt of Compound (I) according to claim 3 having an X-ray powder diffraction pattern further comprising peaks at 18.9, 20.1 and 25.1 degrees 2θ (±0.2 degrees 2θ) when measured using CuKα radiation.

5. The crystalline hemi-succinate salt of Compound (I) according to claim 4 having an X-ray powder diffraction pattern further comprising peaks at 9.0, 22.8, 26.1 and 29.9 degrees 2θ (±0.2 degrees 2θ) when measured using CuKα radiation.

6. The crystalline hemi-succinate salt of Compound (I) according to claim 2 having an X-ray powder diffraction pattern substantially the same as that shown in FIG. 1.

7. The crystalline hemi-succinate salt of Compound (I) according to claim 2 having a DSC thermogram substantially the same as that shown in FIG. 2.

8. The crystalline hemi-succinate salt of Compound (I) according to claim 2 having a TGA curve substantially the same as that shown in FIG. 4.

9. The crystalline hemi-succinate salt of Compound (I) according to claim 2 having an X-ray powder diffraction pattern comprising peaks at 7.1, 10.3 and 12.5 degrees 2θ (±0.2 degrees 2θ) when measured using CuKα radiation and having a DSC thermogram substantially the same as that shown in FIG. 2.

10. The crystalline hemi-succinate salt of Compound (I) according to claim 2 having an X-ray powder diffraction pattern comprising peaks at 7.1, 10.3 and 12.5 degrees 2θ (±0.2 degrees 2θ) when measured using CuKα radiation and having a TGA curve substantially the same as that shown in FIG. 4.

11. A pharmaceutical composition comprising a hemi-succinate salt of Compound (I) according to claim 1, and at least one pharmaceutically acceptable carrier or diluent.

12. The pharmaceutical composition according to claim 11 further comprising at least one other antiviral agent.

13. A method of treating or preventing an HIV infection in a human having or at risk of having the infection by administering to the human a therapeutically effective amount of a hemi-succinate salt of Compound (I) according to claim 1 or a pharmaceutical composition according to claim 11.

14. (canceled)

15. A process to prepare crystalline Form A of the hemi-succinate salt of Compound (I) according to claim 2 comprising the following steps:
(i) dissolving Compound (I) in a suitable solvent to obtain a mixture;
(ii) slowly heating the mixture of step (i) with stirring to a temperature to obtain a solution or slurry;
(iii) slowly adding succinic acid to the solution or slurry of step (ii) to obtain a solution;
(iv) optionally slowly adding a solution of sodium hydroxide to the solution of step (iii);
(v) optionally slowly cooling the solution of the preceding step;
(vi) optionally adding a seed comprising a hemi-succinate salt of Compound (I) to the solution of the preceding step to obtain a slurry;
(vii) optionally slowly adding a solution of sodium hydroxide to the solution or slurry of the preceding step;
(viii) cooling the mixture of the preceding step; and
(ix) collecting the solid material obtained in step (viii) to obtain the hemi-succinate salt of Compound (I).
